(19) United States
(12) Patent Application Publication
(10) Pub. No.: US 2004/0146514 A1
(43) Pub. Date: Jul. 29, 2004

(54) Cancer therapy using multiple antibodies from different species directed against the tumor3

(76) Inventors: James R. Smith, Aliso Viejo, CA (US); Henry J. Smith, San Jacinto, CA (US)

Correspondence Address: HENRY J. SMITH, 2083 E. WASHINGTON AVE., SAN JACINTO, CA, 92583, US

(21) Appl. No.: 10/759,828

(22) Filed: Jan. 20, 2004

Related U.S. Application Data
(60) Provisional application No. 60/441,024, filed on Jan. 21, 2003.

(51) Int. Cl. A61K 39/395

(52) U.S. Cl. 424/155.1

(57) ABSTRACT

This invention describes a method whereby antitumor antibodies obtained from different species and directed against a variety of antigens present in tumors can be used for immunotherapy of cancer. Some of these antibodies may have a direct inhibitory effect upon the tumor, or they may labeled with radionuclides or cytotoxic agents and used as “carriers” to transport the cytotoxic agent to the tumor where they will have maximum effect. By employing a succession of antitumor antibodies produced from different species the risk of the cancer patient developing an allergic reaction to the foreign antibodies is minimized.

### CROSS-REFERENCE TO RELATED APPLICATIONS

This application references U.S. provisional patent application #60/441,024 filed Jan. 21, 2003 and titled “CANCER THERAPY USING MULTIPLE ANTIBODIES FROM DIFFERENT SPECIES DIRECTED AGAINST THE TUMOR”.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

One out of every four people in the US will die from cancer. Current methods of cancer chemotherapy utilize cytotoxic drugs that are effective against cancer cells but also kill normal cells. There is intensive research into methods for increasing the tumor cytotoxic effect and reducing the serious side effects. One promising method is to use antitumor antibodies to target the tumor and to combine these antibodies with cytotoxic agents to selectively deliver them to the tumor where they will have the most effect.

Early research on targeting tumors used antibodies prepared in animals immunized against tumor antigens. More recent studies have focused on using monoclonal antitumor antibodies prepared in murine hybridomas.

There is a problem however, when animal proteins are injected into cancer patients. The patient may develop an immune response against the “foreign” antibody making further treatment ineffective. In order to mitigate this problem, there is intensive research into developing “humanized” antibodies by replacing parts of the murine monoclonal antibody with human components using genetic engineering methods. Other methods of producing fully human antibodies include using human hybridomas or using transgenic animals whose immune system has been replaced with human antibody producing cells. All of these methods are complex and difficult to perform.

This invention describes an alternative approach of targeting tumors that does not involve procedures for “humanizing” or altering the composition of the antibody molecule. Instead a systematic treatment protocol is described in which the cancer patient is treated with a succession of antitumor antibodies prepared in different species of animals. This limits the exposure of the patient to repeated challenge to any particular animal species protein and minimizes the risk of the patient developing a severe allergic reaction to the “foreign” proteins.

It is a well-established immunological principle that when a patient is first injected with a foreign protein the patient develops an immune response to the “foreign” protein that is generally mild and self-limiting in nature. However, the patient could become “sensitized” to that particular foreign protein, and if the patient then receives a later injection of the same antigen a more severe allergic reaction may develop. This invention teaches that this severe secondary allergic reaction can be avoided if the patient is treated with a succession of antitumor antibodies prepared in different species of animals and does not receive more than one injection of an antitumor antibody that is prepared in a particular animal species.

The antitumor antibodies can be used alone or labeled with a variety of radionuclides and/or cytotoxic pharmaceuticals and used as “carriers” to transport these agents to the tumor site.

A further advantage of using this approach is that it is possible to target multiple antigens on the tumor and to design individualized treatment protocols for each cancer patient.

### SUMMARY OF THE INVENTION

This invention teaches the sequential use of multiple antitumor antibodies prepared in different animal species to target the tumor. By limiting the cancer patient's exposure to any particular animal species antibody to a single exposure only, the risk of the patient developing a severe allergic reaction when exposed to antibody from a different species is minimized.

Different species of animals are immunized with tumor extracts to produce antitumor antibodies. The antibodies are directed against one or more antigens possessed by the tumor. For example, these include tumor antigens and/or antigens present in both tumor and normal cells. In this invention the term “tumor antigen” is used to describe all varieties of antigens that are found in tumor cells including those also shared by normal cells. The antitumor sera are produced and purified according to conventional laboratory techniques. The antisera can be used alone to target the tumor or they can be labeled with a variety of radionuclides and cancer drugs and used to carry the anticancer agent to the tumor where it will have the most effect.

The cancer patient receives a succession of treatments with each treatment using antitumor antibodies prepared from a different animal species. As the patient is exposed to a particular foreign antigen only once, there is little risk that the patient will develop a severe allergic reaction when treated with an unrelated foreign antibody. As a further safety precaution the patient is also pre-tested for immune reactivity to the antitumor antibody using standard laboratory tests such as the enzymelinkedimmunosorbant assay (ELISA) to test serum samples of the patient. In addition the patient is also skin tested against the species immunoglobulin. Only those preparations that were non-reactive in laboratory tests and in skin testing are used for treatment.

The cancer treatment may consist of the antisera used alone or combined with radionuclides or cancer drugs and used as a carrier to transport the drug to the tumor site. There are a number of different ways that the drug:antibody combinations can be used therapeutically. For example: a single pharmaceutical is linked to different species antibodies and used to target a single tumor antigen; and/or a single pharmaceutical is linked to different species antibodies and used to target multiple tumor antigens; and/or different pharmaceuticals are linked to different species antibodies and used to target a single tumor antigen; and/or different pharmaceuticals are linked to different species antibodies and used to target multiple tumor antigens.

### DESCRIPTION OF THE INVENTION

This invention teaches the use of antitumor antibodies that are prepared in multiple species of animals in the treatment of cancer. It describes the treatment protocols that are employed to ensure that the cancer patient does not develop a severe allergic to the injected antibodies; and it also describes the various methods whereby the antitumor antibodies can be used alone or combined with anti-cancer drugs and used to transport them to the tumor site.

“Tumor Antigens”

In this invention the term “tumor antigen” is used in the broadest sense to describe all varieties of antigens that are found in tumor cells including those also shared by normal cells. These include the large number of tumor associated antigens reported in the literature such as CEA, fetoprotein, Her2 protein, epidermal growth factor receptor and other tumor expressed proteins; cell surface marker proteins such as cluster determinants (CD) markers found on both tumor cells and normal cells; and intracellular material released from dead tumor cells. For example tumor cells contain intracellular components that are released when the tumor cell dies and this expressed material can also become targets for immunotherapy. Many tumors have areas of necrosis and these necrotic areas contain elevated levels of intracellular material released from dead or dying cells. This includes nuclear materials such as the nuclear membrane, nucleoproteins, DNA, histones etc. and cytoplasmic components such as mitochondria, ribosomes and soluble cytoplasmic proteins. Other examples of expressed intracellular material include melanin released from dead melanoma cells and myosin released from dead sarcoma cells.

Antisera Preparation

Different species of animals are immunized with tumor extracts to produce antitumor antibodies. The species of animals used include: horse, donkey, cow, goat, sheep, rabbit, turkey, chicken, rat, mice and other animal species. The animals are immunized according to standard laboratory procedures to produce polyclonal antitumor antisera. Monoclonal antitumor antibodies using rat or murine hybridomas may also be produced using standard laboratory procedures. In certain circumstances human autoantibodies may be employed to target intracellular antigens present within tumors.

The methods of preparing tumor antigens and immunizing different animal species are known to those skilled in the art. For example, the tumor is homogenized and fractionated using centrifugation, gel-filtration and other separation techniques. The tumor fractions are used to immunize animals by injecting them with the material incorporated in Freund's complete adjuvant followed by a booster injection several weeks later of the same material given alone or incorporated in Freund's incomplete adjuvant. The animals are periodically bled and tested for activity against the tumor antigen using standard laboratory tests such as ELISA. Once the antisera show a good titer and specificity against the tumor antigen of interest it is purified using standard laboratory techniques such as ammonium sulphate precipitation, gel-filtration and affinity binding techniques. Other methods of antigen preparation and antibody production, known to those skilled in the art, may also be employed and are considered within the scope of this invention.

Monoclonal antitumor antibodies may be produced in murine or rat hybridomas using standard production methods. The monoclonal antibodies are screened for specific tumor activity and purified using affinity binding techniques.

In certain circumstances human autoantibodies directed against intracellular material such as nuclear antigens may be used. For example, patients with SLE have antibodies against nuclear antigens. The antinuclear antibodies can be purified using standard purification techniques such as gel-filtration and affinity binding.

Animals immunized with tumor antigens will produce IgM and IgG antibodies against the tumor antigen. In some cases it would be preferable to use the binding fragments Fab and F(ab)2 of the antibody molecule. The Fab and F(ab)2 binding fragments being of smaller size may be able to penetrate more rapidly into the tumor tissue and also the unbound material may be more rapidly excreted from the body. Another advantage in using the binding fragments is that they may be less antigenic than the complete antibody molecule. In this invention the term “antitumor antibodies” includes the IgM antibodies, the IgG antibodies and the Fab and Fab2 binding fragments of the antibodies.

The purified antibodies are used alone or combined with a variety of pharmaceutical compounds and used for cancer treatment.

Antitumor Antibodies Used Alone

Purified antitumor antibodies from different species are prepared as described above. Cancer patients are first pre-tested for immune reactivity to the antibody that will be injected. Standard laboratory tests such as ELISA are used before each treatment to determine immune reactivity and only patients who are non-reactive to that species antibody will receive that antibody. In addition, the patient will be skin tested with an extremely dose of the selected species antibody shortly before the scheduled treatment (i.e. 1-24 hrs before treatment) in order to confirm that the patient is non-reactive to that species antibody. The amount of antibody injected will vary depending upon the characteristics of the tumor e.g. tumor type, size, degree of malignancy, the target antigen and the particular antibody being employed. The antibody alone may have an inhibitory effect upon the tumor e.g. antibody directed against the Her2 protein or antibody directed against epidermal growth factor receptor. There may also be a secondary inhibitory effect upon the tumor resulting from the patient's immune response against the foreign antibody protein bound to the tumor cells.

The following examples are provided for illustrative purposes only. The actual treatment protocol and the antisera employed will vary depending upon numerous factors including tumor type, size, malignancy and the patient's condition. For example, the patient receives an injection of horse antitumor antibody directed against the Her 2 protein. The patient develops an immune reaction to the horse protein bound to the tumor which may inhibit tumor growth. The patient later receives an injection of goat antitumor antibody directed against the HER 2 protein or another tumor antigen. The patient develops an immune reaction to the goat protein bound to the tumor which may inhibit tumor growth. Other species antibodies directed against other tumor antigens may be similarly employed and are considered to be within the scope of this invention.

Antitumor Antibodies used as Carriers for Pharmaceuticals

In order to increase the tumor inhibitory effect the anti-tumor antibodies are labeled with a variety of cytotoxic compounds and used to transport these agents to the tumor site. The pharmaceutical compounds that can be linked to the carrier antibodies can be classified into the following groups:

The radiologic group includes alpha-emitting and beta-emitting radionuclides such as I-131, Yt-99, Cu-67, Au-198, P-32, and other cytotoxic radionuclides. The radionuclides are conjugated to the carrier antibody using methods that are familiar to those skilled in the art. For example, the carrier protein can be iodinated using the chloramine-T method to label the protein with I-125 or. 1-131. Other radionuclides may be attached to the carrier antibody by chelation with benzyl EDTA or DPTA conjugation procedures. The labeled carrier protein is then injected into the cancer patient where it will bind to the target antigens. From there the radiation will penetrate into the surrounding tumor where its will have a cytotoxic effect upon the tumor cells.

The cytotoxic drug group includes the folate inhibitors, pyrimidine analogs, purine analogs, alkylating agents and antibiotics. Specific examples include acivicin, aclarubicin, acodazole, adriamycin, ametantrone, aminoglutethimide, anthramycin, asparaginase, azacitidine, azetepa, bisantrene, bleomycin, busulfan, cactinomycin, calusterone, caracemide, carboplatin, carmustine, carubicin, chlorambucil, cisplatin, cyclophosphamide, cytarabine, dacarbazine, dactinomycin, daunorubicin, dezaguanine, diaziquone, doxorubicin, epipropidine, etoposide, etoprine, floxuridine, fludarabine, fluorouracil, fluorocitabine, hydroxyurea, iproplatin, leuprolide acetate, lomustine, mechlorethamine, megestrol acetate, melengestrol acetate, mercaptopurine, methotrexate, metoprine, mitocromin, mitogillin, mitomycin, mitosper, mitoxantrone, mycophenolic acid, nocodazole, nogalamycin, oxisuran, peliomycin, pentamustine, porfiromycin, prednimustine, procarbazine hydrochloride, puromycin, pyrazofurin, riboprine, semustine, sparsomycin, spirogermanium, spiromustine, spiroplatin, streptozocin, talisomycin, tegafur, teniposide, teroxirone, thiamiprine, thioguanine, tiazofurin, triciribine phosphate, triethylenemelamine, trimetrexate, uracil mustard, uredepa, vinblastine, vincristine, vindesine, vinepidine, vinrosidine, vinzolidine, zinostatin and zorubicin. Also included are the toxins such as ricin and diptheria toxin.

All these compounds can be conjugated to the carrier antibody using methods that are familiar to those skilled in the art. For example, many carboxylic acid-containing compounds such as methotrexate can be conjugated to immunoglobulins through an active ester intermediate by reacting the compound with N-hydroxysuccinimide and dicyclohexylcarbodiimide; amino sugar containing drugs such as adriamycin and daunomycin may be covalently bound to antibodies by periodate oxidation of the drug, followed by linking of the oxidized drug to the immunoglobulin and subsequent reduction of the product with sodium borohydride. The methods of conjugating any particular drug to the carrier protein will vary depending upon the nature of the drug. However, these are according to conventional laboratory methods and are considered to be within the scope of this invention.

The labeled carrier protein is then injected into the cancer patient where it will bind to the tumor antigens.

The biological response modifier group includes cytokines such as tumor necrosis factor, interferons, angiostatin and immune stimulators such as animal or microbial proteins. These compounds can be conjugated to the carrier antibody using methods that are familiar to those skilled in the art. For example, glutaraldehyde may be used to cross-link the free amino groups of the antibody and modifier protein. Other methods may be employed using conventional laboratory procedures and are considered to be within the scope of this invention.

The labeled carrier protein is then injected into the cancer patient where it will bind to the tumor antigens. The effect may be to stimulate an inflammatory response as in the case of tumor necrosis factor, or to inhibit the growth of new blood vessels to the tumor as in the case of angiostatin, or to stimulate an immune response within the tumor by the foreign animal or microbial protein.

Cancer Treament Protocol

The cancer patient receives a succession of treatments using antitumor antibodies obtained from different species. This minimizes the risk of the patient reacting to antibodies of other species. As a further precaution the patient is pre-tested for immune reactivity to the carrier antibody using standard laboratory tests such as the enzymelinkedimmunoabsorban assay (ELISA) and only preparations that are non-reactive are used for treatment. The cancer treatment may consist of a single pharmaceutical linked to different species antibodies directed against a specific antigen; a single pharmaceutical linked to different species antibodies directed against multiple antigens; different pharmaceuticals linked to different species antibodies directed against a specific antigen; and different pharmaceuticals linked to different species antibodies directed against multiple antigens.

Depending upon the particular type of tumor the patient's condition the treatment protocol can be individualized by a “mix and match” permutation of the procedures outlined above. For example, for certain tumors responding to a particular drug such as methotrexate it may be advantageous to target multiple tumor antigens using antibody from different species each labeled with methotrexate; while for other tumors it may be preferable to target one type of tumor antigen with one cancer drug such as methotrexate and to target a different type of tumor antigen within the same tumor with a different cancer drug such as doxorubicin. These examples below are presented for illustrative purposes only. There are a very large number of different treatment permutations possible and these are all considered to be within the scope of this invention.

### EXAMPLE 1

### Carrier Antibody against a Single Cancer Antigen Prepared in Different Species and Labeled with One Type of Anti-Cancer Pharmaceutical

To illustrate this principle antibodies against the tumor associated HER 2 protein are prepared in different species of animals such as horse, goat, rabbit, chicken etc. The carrier antibody from each species is labeled with the drug paclitaxel. The cancer patient receives a succession of paclitaxel labeled carrier antibodies prepared in different species. For example, the first treatment may be paclitaxel labeled horse antibody; the second treatment may be paclitaxel labeled goat antibody; the third treatment may be paclitaxel labeled rabbit antibody; and the fourth treatment may be paclitaxel labeled chicken antibody etc. In this manner the patient is not exposed to a particular species antibody more than once, and therefore the patient will not develop an allergic response to the foreign proteins. It is apparent from this principle that different types of tumor can be targeted; that different species of animals can be used for antibody production; that a different cancer pharmaceutical may be used for labeling; and that the sequence of species antibody used as carriers can be changed without affecting the novelty of this invention.

### EXAMPLE 2

### Carrier Antibody against a Single Cancer Antigen Prepared in Different Species and Labeled with Different Types of Anti-Cancer Pharmaceuticals

To illustrate this principle antibodies against the tumor associated HER 2 protein are prepared in different species of animals such as horse, goat, rabbit, chicken etc. The carrier antibody from one species is labeled with the drug paclitaxel and the carrier antibody from a different species is labeled with the drug methotrexate; and the carrier antibody from a third species is labeled with a radionuclide such as I¹³¹. The cancer patient receives a succession of carrier antibodies from different species labeled with different cytotoxic agents. For example, the first treatment may be paclitaxel labeled horse antibody against the HER 2 protein; the second treatment may be methotrexate labeled goat antibody against the HER 2 protein; the third treatment may be I¹³¹ labeled rabbit antibody against the HER 2 protein; In this manner the patient is not exposed to a particular species antibody more than once and therefore the patient will not develop an allergic response to the foreign proteins. It is apparent from this principle that different types of tumor can be targeted; that different species of animals can be used for antibody production; that a series of different cancer pharmaceuticals may be used for labeling; and that the sequence of species antibody used as carriers can be changed without affecting the novelty of this invention.

### EXAMPLE 3

### Carrier Antibodies against Multiple Tumor Antigens Prepared in Different Species and Labeled with One Type of Anti-Cancer Pharmaceutical

To illustrate this principle antibodies against the tumor associated HER 2 protein are prepared in one species of animals such as the horse; and antibodies against a different tumor antigen such as epidermal growth factor receptor are prepared in goats, and antibodies against a different tumor antigen such as intracellular nuclear antigens are prepared in rabbits etc. The carrier antibody from each species is then labeled with a particular anti-cancer drug. The cancer patient receives a succession of drug labeled carrier antibodies from different species. For example, the first treatment may be paclitaxel labeled horse antibody directed against the HER 2 protein; the second treatment may be paclitaxel labeled goat antibody directed against growth factor receptor; the third treatment may be paclitaxel labeled rabbit antibody directed against extracellular nuclear antigens etc. In this manner the patient is not exposed to a particular species antibody more than once and therefore the patient will not develop an allergic response to the foreign proteins. It is apparent from this principle that a variety of tumor associated antigens can be used to target different types of tumor; that different species of animals can be used for antibody production; that different cancer pharmaceuticals may be used for labeling; and that the sequence of species antibody used as carriers can be changed without affecting the novelty of this invention.

### EXAMPLE 4

### Carrier Antibodies against Multiple Cancer Antigens Prepared in Different Species and Labeled with Different Types of Anti-Cancer Pharmaceuticals

To illustrate this principle antibodies against the tumor associated HER 2 protein are prepared in one species of animals such as the horse; and antibodies against a different tumor antigen such as growth factor receptor are prepared in goats, and antibodies against a different tumor antigen such as intracellular nuclear antigens are prepared in rabbits etc. The carrier antibody from each species is then labeled with a different anti-cancer drug. The cancer patient receives a succession of different drug labeled carrier antibodies from different species. For example, the first treatment may be paclitaxel labeled horse antibody directed against the HER 2 protein; the second treatment may be methotrexate labeled goat antibody directed against growth factor receptor; the third treatment may be I¹³¹ labeled rabbit antibody directed against extracellular nuclear antigens etc. In this manner the patient is not exposed to a particular species antibody more than once and therefore the patient will not develop an allergic response to the foreign proteins. It is apparent from this principle that a variety of tumor associated antigens can be used to target different types of tumor; that different species of animals can be used for antibody production; that different cancer pharmaceuticals may be used for labeling; and that the sequence of species antibody used as carriers can be changed without affecting the novelty of this invention.

In a further embodiment of this invention the antitumor antibodies bound to the tumor antigens within the tumor can themselves become a target for subsequent antibody based pharmaceuticals. Many tumor antigens are antigenically weak and this method of pre-targeting the tumor provides a means whereby the foreign antibody bound to the tumor provides an amplified antigenic signal for subsequent antibody based pharmaceuticals. For example, the cancer patient receives a first injection of antitumor antibody prepared in an animal species such as goat. The goat antitumor antibodies will bind to the tumor antigens and become fixed at the tumor site while any unbound goat proteins are eliminated from the patient's normal tissues over time. The patient now receives a second injection of antibody prepared in a different species of animal such as rabbit, but in this instance the rabbit antibody is directed towards goat immunoglobulin. As the only remaining goat antibodies are those bound within the tumor the rabbit antibody will in turn become bound to the goat protein fixed within the tumor and not to normal tissues. The rabbit antibody is labeled with a radionuclide or cancer drug and used to transport the cytotoxic agent to the tumor site where it will have maximum effect. It is apparent from this principle that a variety of tumor associated antigens can be used to target different types of tumor; that different species of animals can be used for antibody production against the species immunoglobulin of the pre-targeting antibody; that different cancer pharmaceuticals may be used for labeling the carrier antibody; and that the sequence of species antibody used as carriers can be changed without affecting the novelty of this invention.

What is claimed is:

1. A process for cancer immunotherapy that utilizes a succession of antitumor antibodies prepared from different species.

2. According to claim 1 the species of animals used to prepare the antitumor antibodies include: horse, donkey, cow, goat, sheep, rabbit, turkey, chicken, rat, mice and other animal species including human autoantibodies.

3. According to claim 1 the term “tumor antigen” includes all types of antigen found in tumors including those shared by normal cells such as tumor associated antigens, cluster determinant (CD) markers, and intracellular components such as nuclear and cytoplasmic material released by dead tumor cells into the surrounding environment.

4. According to claim 1 the term “antitumor antibody” includes all types of polyclonal and monoclonal antibodies.

5. According to claim 5 the antitumor antibodies may consist of the whole IgG molecule or the whole IgM molecule or the binding Fab and F(ab)2 fragments of the antibody.

6. According to claim 1-5 the cancer patient is pre-tested by laboratory testing and by skin testing against the species animal immunoglobulin to determine non-reactivity before treatment with the antitumor antibody.

7. According to claims 1-6 a process of cancer treatment utilizing a therapeutic dosage of a variety of radionuclides linked to carrier antitumor antibodies from different species which is injected into the cancer patient.

8. According to claims 1-6 a process of cancer treatment utilizing a variety of cytotoxic anti-cancer drugs linked to carrier antitumor antibodies from different species which is injected into the cancer patient.

9. According to claims 1-6 a process of cancer treatment utilizing a variety of biological response modifiers linked to carrier antitumor antibodies which is injected into the cancer patient.

10. According to claims 1-6 a process of cancer treatment utilizing a variety of toxins linked to carrier antitumor antibodies which is injected into the cancer patient.

11. According to claims 1-6 a process of cancer treatment utilizing a variety of blood vessel growth inhibiting compounds linked to carrier antitumor antibodies which is injected into the cancer patient.

12. According to claims 1-11 a process of cancer treatment whereby the cancer patient receives a single pharmaceutical linked to different species antibodies directed against a specific antigen.

13. According to claims 1-11 a process of cancer treatment whereby the cancer patient receives a single pharmaceutical linked to different species antibodies directed against multiple antigens.

14. According to claims 1-11 a process of cancer treatment whereby the cancer patient receives different pharmaceuticals linked to different species antibodies directed against a specific antigen.

15. According to claims 1-11 a process of cancer treatment whereby the cancer patient receives different pharmaceuticals linked to different species antibodies directed against multiple antigens.

16. According to claims 1-6 a process of cancer treatment whereby the cancer patient receives a pre-targeting injection of antitumor antibody from one animal species, followed by later injections of radionuclide labeled antibody and/or drug labeled antibody prepared in a different species and directed against the immunoglobulin component of the first animal species.

17. According to claims 1-6 a process of cancer treatment whereby the cancer patient is only exposed once to the antibody from a particular animal species which minimizes the risk of the patient developing an allergic reaction to the therapeutic antibodies.
